(19) United States
(12) Patent Application Publication
(10) Pub. No.: US 2025/0213321 A1
(43) Pub. Date: Jul. 3, 2025

(54) APPARATUS FOR MEASURING INSERTION DEPTH OF MEDICAL INSTRUMENTS INSERTED INTO THE BODY AND METHOD FOR MEASURING INSERTION DEPTH OF MEDICAL INSTRUMENTS INSERTED INTO THE BODY USING THE SAME

(71) Applicants: Jongwoon Choi, Seoul (KR); DSE Inc., Seoul (KR)

(72) Inventor: Jongwoon Choi, Seoul (KR)

(21) Appl. No.: 18/985,631

(22) Filed: Dec. 18, 2024

(30) Foreign Application Priority Data
Dec. 27, 2023 (KR) 10-2023-0192460

(51) Int. Cl. A61B 90/00; A61B 1/00; A61B 1/05; A61B 17/00; A61B 18/00; A61B 18/20

(52) U.S. Cl. CPC A61B 90/06; A61B 1/00142; A61B 1/05; A61B 18/20; A61B 2017/0003; A61B 2018/00559; A61B 2018/00982; A61B 2090/062

(57) ABSTRACT

The present disclosure relates to an apparatus for measuring an insertion depth of medical instruments inserted into a body and a method for measuring an insertion depth of medical instruments inserted into a body. The apparatus for measuring an insertion depth of medical instruments inserted into a body includes: a non-metallic protective container that is inserted into a body with a medical instrument disposed therein; a pair of main electrodes having a predetermined area and formed in a longitudinal direction while facing each other in the protective container; and a controller electrically connected to the pair of main electrodes, measuring capacitance that is induced by the pair of main electrodes, and calculating an insertion depth of the medical instrument inserted into a body on the basis of the measured capacitance.

### CROSS-REFERENCE TO RELATED APPLICATION

This application claims the benefit of priority under 35 U.S.C. § 119(a) to Korean Patent Application No. 10-2023-0192460, filed on Dec. 27, 2023, the entire contents of which are incorporated herein by reference.

### BACKGROUND

### (a) Technical Field

The present disclosure relates to an apparatus for measuring an insertion depth of medical instruments inserted into a body and a method for measuring an insertion depth of medical instruments inserted into a body using the same, and more specifically to an apparatus and method for electrically measuring an insertion depth of a medical instrument inserted into a body from the surface of the skin when inserting the medical instrument into the body.

### (b) Background Art

Female genital surgical instruments using a fractional CO₂ laser has been steadily gaining attention in the field of obstetrics and gynecology. This is because it has been confirmed that there is an effect that heat shock protein 70 (HSP 70) by micro-thermal injury is increased through a skin remodeling process by stimulation on collagen due to laser emission, collagen protein and capillaries are increased, and the vaginal wall becomes firmer and thicker.

Currently, when inserting a female genital surgical instrument into the vagina, the insertion depth is adjusted by an operator visually checking the scale marked on the surface of the instrument. The method of relying on the operator's visual inspection makes it difficult to accurately adjust the depth during the use of surgical instruments, so there are often case where a procedure is repeated or parts are left untreated.

Further, due to the nature of performing surgery on the inside of a body, there is a problem in that there is no method to record or verify a procedure process and its results.

### PRIOR ART DOCUMENT

### [Patent Document]

Korean Patent No. 10-1855743

### SUMMARY OF THE DISCLOSURE

It is an object of the present disclosure to solve the problems described above and additional problems. It is another object to provide an apparatus for measuring an insertion depth of medical instruments inserted into a body and a method for measuring an insertion depth of medical instruments inserted into a body using the same.

In order to achieve the above-mentioned or additional objects, according an aspect of the present disclosure, there may be provided an apparatus for measuring an insertion depth of medical instruments inserted into a body, the apparatus including: a non-metallic protective container that is inserted into a body with a medical instrument disposed therein; a pair of main electrodes having a predetermined area and formed in a longitudinal direction while facing each other in the protective container; and a controller electrically connected to the pair of main electrodes, measuring capacitance that is induced by the pair of main electrodes, and calculating an insertion depth of the medical instrument inserted into a body on the basis of the measured capacitance.

According to an aspect of the present disclosure, the apparatus may further include a pair of reference electrodes disposed on extension lines of the main electrodes, formed electrically separately from the main electrodes, facing each other, and having a preset length, wherein capacitance that is induced at the reference electrodes may be measured by the controller.

According to an aspect of the present disclosure, the pair of main electrodes and the pair of reference electrodes may be made of the same material in the same shape.

According to an aspect of the present disclosure, when the pair of reference electrodes is fully inserted into a body and the pair of main electrodes is partially inserted into the body and when a length of the reference electrodes is B, capacitance that is induced at the reference electrodes is C1, a length of the section of the main electrodes inserted into the body is X, and capacitance at the section of the main electrodes inserted in the body is C2, the following equation may be satisfied:

$$X = ( {B}^{*} C 2 ) / C 1$$

According to an aspect of the present disclosure, the main electrodes and the reference electrodes may have a curved surface or flat surface shape.

According to an aspect of the present disclosure, the protective container may be an instrument that treats the inside of a body, and an endoscopic camera may be disposed at a front end inside the protective container.

According to an aspect of the present disclosure, a disc-shaped insertion separation plate may be disposed on a circumference of the protective container.

According to an aspect of the present disclosure, the protective container may have a laser output window, so a laser may be output to the outside through the window.

According to another aspect of the present disclosure, there may be provided a method for measuring an insertion depth of medical instruments inserted into a body, the method including the steps of: measuring capacitance C1 in a body by a pair of reference electrodes disposed in a non-metallic protective container that is inserted in the body with a medical instrument disposed therein; measuring capacitance C2 induced at a pair of main electrodes disposed on extension lines of the reference electrodes in the protective container; and calculating an insertion depth of the main electrodes in the body using the C1 and C2, wherein the capacitance C2 changes, depending on the insertion depth in the body.

The apparatus for measuring an insertion depth of medical instruments inserted into a body according to the present disclosure and the method for measuring an insertion depth of medical instruments inserted into a body using the same are described as follows.

According to at least one of embodiments of the present disclosure, there is an advantage that it is possible to electrically measure a depth at which a medical instrument that is inserted into a body is inserted inside the skin.

The method used to measure an insertion depth was to mark a scale on the surface of a medical instrument and allow an operator to visually read the scale value in the related art, whereas, according to at least one of embodiments of the present disclosure, there is an advantage that it is possible to easily and accurately measure the insertion depth of a medical instrument in comparison to the method of measuring an insertion depth by an operation visually checking it by installing electrodes in the medical instrument and measuring capacitance that is induced between the electrodes and a body.

According to at least one of embodiments of the present disclosure, there is an advantage that it is possible to electrically record a surgical history related to an insertion depth of a medical instrument, it is possible to measure an insertion depth value by installing capacitor electrodes in an insertion tube of a female genital surgical instrument, and it is possible to apply accurate image records to treatment by adding depth information to each image by combing and using an endoscope.

According to at least one of embodiments of the present disclosure, there is an advantage that an operator can remotely perform a surgical operation such as a robot surgical operation by electrically measuring an insertion depth of a medical instrument inserted into a body.

Additional range of applicability of the present disclosure will be made clear from the following detailed description. However, various changes and modifications within the spirit and scope of the present disclosure can be clearly understood by those skilled in the art, so the detailed description and specific embodiments such as preferred embodiments of the present disclosure should be understood only as examples.

### BRIEF DESCRIPTION OF DRAWINGS

FIG. 1 is a view showing a non-metallic protective container according to an embodiment of the present disclosure and a pair of main electrodes disposed in the protective container.

FIG. 2 is a view showing the pair of main electrodes and reference electrodes disposed in the non-metallic protective container according to an embodiment of the present disclosure.

FIG. 3 is a view illustrating the functions of the main electrodes and the reference electrodes disposed in the non-metallic protective container inserted into a body in accordance with an embodiment of the present disclosure.

FIG. 4 is a view in which the non-metallic protective container according to an embodiment of the present disclosure has been applied to a female genital surgical instrument.

FIG. 5 is a view when the non-metallic protective container according to an embodiment of the present disclosure has been applied to an endoscope.

FIG. 6 is a flowchart of a method for measuring an insertion depth of medical instruments inserted into a body according to an embodiment of the present disclosure.

### DETAILED DESCRIPTION

Hereinafter, embodiments of the present disclosure will be described in detail with reference to the accompanying drawings, and the same or similar components are given the same reference numerals regardless of the numbers of figures and duplicate descriptions thereof will be omitted. The term “unit” that is used for components in the following description is used only for the ease of description without having discriminate meanings or functions in itself. In describing the embodiments disclosed in this specification, if it is determined that the detailed description of known technologies related to the present disclosure may obscure the subject matter of the embodiments described herein, the detailed description is omitted. Further, the accompanying drawings are provided only for easy understanding the embodiments disclosed herein without limiting the technical spirit disclosed herein and should be understood as including all of changes, equivalents, and substitutes included in the spirit and technical scope of the present disclosure.

Singular forms are intended to include plural forms unless the context clearly indicates otherwise.

It will be further understood that the term “comprise” or “have” used in this specification, specifies the presence of features, steps, operations, components, parts described herein, or a combination thereof, but does not preclude the presence or addition of one or more other features, numerals, steps, operations, components, parts, or a combination thereof.

An embodiment of the present disclosure relates to an apparatus 100 for measuring an insertion depth of medical instruments inserted into a body, that is, to an apparatus for measuring the insertion depth of a medical instrument inserted into a body by measuring capacitance that is generated between two electrodes when the medical instrument having the two electrodes is inserted into the body.

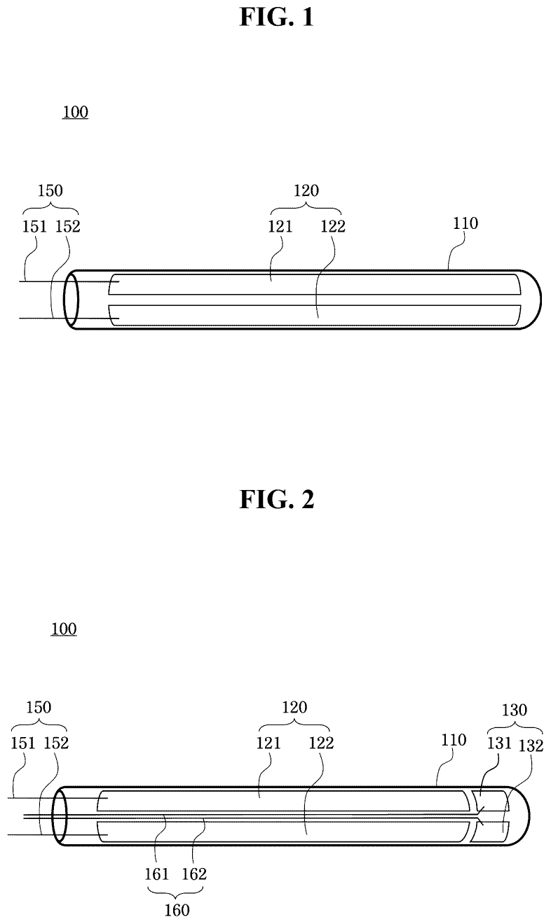

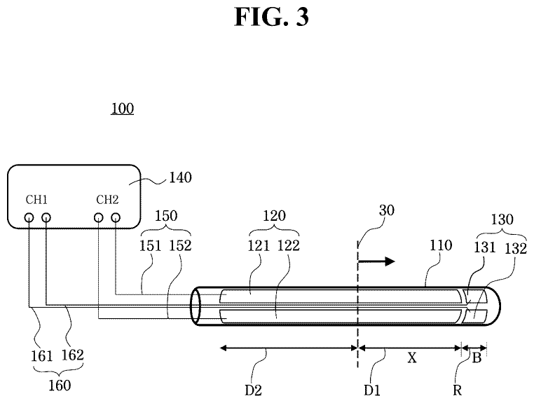

First, FIG. 1 is a view showing a non-metallic protective container 110 according to an embodiment of the present disclosure and a pair of main electrodes 120 disposed in the protective container 110, FIG. 2 is a view showing the pair of main electrodes 120 and reference electrodes 130 disposed in the non-metallic protective container 110 according to an embodiment of the present disclosure, and FIG. 3 is a view illustrating the functions of the main electrodes 120 and the reference electrodes 130 disposed in the non-metallic protective container 110 inserted into a body in accordance with an embodiment of the present disclosure. The main electrodes 120 and the reference electrodes 130 that are capacitor electrode are made of thin copper, a flexible printed circuit board, or an aluminum foil, and can be attached to the protective container 110 by a double-sided tape.

In more detail, FIG. 1 is a view showing the internal configuration of the apparatus 100 for measuring an insertion depth of medical instruments inserted into a body using a non-metallic protective container covering a medical instrument that is inserted into a body, and FIG. 2 is a view illustrating a reference electrode 130 that is used to minimize the influence by the surrounding situation and the measurement target body. Further, FIG. 3 is a view showing the principle of measuring the insertion depth of a surgical instrument inserted into a body using capacitance.

Thereafter, the apparatus 100 for measuring an insertion depth of medical instruments inserted into a body according to an embodiment of the present disclosure is described with reference to FIG. 1 to FIG. 3.

The apparatus 100 for measuring an insertion depth of medical instruments inserted into a body according to an embodiment of the present disclosure includes: a non-metallic protective container 110 that is inserted into a body with a medical instrument disposed therein; a pair of main electrodes 121 and 122 having a predetermined area and formed in a longitudinal direction while facing each other in the protective container 110; and a controller 140 electrically connected to the pair of main electrodes 121 and 122, measuring capacitance that is induced by the pair of main electrodes 121 and 122, and calculating the insertion depth inserted into a body on the basis of the measured capacitance. In this configuration, the pair of main electrodes 120 is composed of a first main electrode 121 and a second main electrode 122.

The apparatus 100 for measuring an insertion depth of medical instruments inserted into a body according to an embodiment of the present disclosure includes a protective container 110 protecting a medical instrument, a pair of capacitor electrodes that is main electrodes 120, and a controller 140 provided outside the protective container 110 and controlling the medical instrument. The protective container 110 has a medical instrument insertion hole formed at an end so that a medical instrument can be inserted therein, and has another end that is sealed and is formed with a curved surface to reduce friction when it is inserted into a body. The protective container 110 can be inserted into a body, particularly, into the female vagina, and the insertion direction is referred to as a longitudinal direction.

The protective container 110 is made of non-metal and capacitance induced at the main electrodes 120 reflects permittivity of a body around the electrodes inserted in the body. The main electrode 120 is at least partially inserted into a body and the non-inserted portions of the main electrodes 120 are exposed to the air, so capacitance induced at the portions of the main electrodes 120 not inserted into the body reflects the permittivity of the air.

Referring to FIG. 1, assuming that two metallic electrodes having an area A and disposed in parallel are attached with a distance d therebetween in a cylindrical protective container 110 as the pair of main electrodes 121 and 122 shown in FIG. 1, approximate capacitance C between the pair of main electrodes 121 and 122 can be obtained as in the following equation (1).

$$C = ( {{ε}_{0}}^{*} {ε}_{r} ) A / d \qquad ( 1 )$$

wherein the C is capacitance, A is the overlap area of the pair of main electrodes 121 and 122, d is the distance between the pair of main electrodes 121 and 122, εᵣ is relative permittivity, and ε₀ is vacuum permittivity.

The main electrodes 120 are connected to the controller 140 by main electrode connection wires 150, and the first main electrode 121 and the second main electrode 122 are connected to a first main electrode connection wire 151 and a second main electrode connection wire 152, respectively.

In the equation (1), the area A is calculated as the product of the width and the length of the main electrodes 120 that are metallic electrodes. Capacitance that is induced at the metallic electrode before the protective container 110 is inserted into a body is determined by vacuum permittivity by the equation (1). When the protective container 110 with the metallic electrode installed therein is inserted into a body, the body permittivity εᵣ around the metallic electrodes is applied, whereby capacitance is determined. Accordingly, by measuring capacitance that is induced at parallel metallic electrodes, the insertion depth of the protective container 110 inserted into a body can be measured.

Meanwhile, the permittivity of a body around electrodes inserted into a body may be differently shown, depending on people. In order to offset this variable, the reference electrodes 130 are used in an embodiment of the present disclosure.

That is, a pair of reference electrodes 140 having a preset known length is formed to face each other on the extension lines of the main electrodes 120 and capacitance that is induced at the reference electrodes 130 is measured by the controller 140. The reference electrodes 130 are composed of a first reference electrode 131 and a second reference electrode 132. In more detail, as in FIG. 2, capacitance that is induced while the pair of reference electrodes 130 facing each other and having an accurate known length is inserted first into a body is a value reflecting permittivity of the body. In this case, the capacitance that is induced at the assistant electrodes 130 is applied with the same permittivity for the capacitance that is induced at the main electrodes 120. Accordingly, by using the reference electrodes 130, it is possible to measure capacitance while minimizing the influence by external permittivity. That is, it is possible to measure the insertion depth of the main electrodes 120 inserted into a body by comparing the capacitance that is induced at the reference electrodes 130 having a known length and the capacitance that is induced at the main electrodes 120. The insertion depth inserted into a body by the capacitances at the main electrodes 120 and the reference electrodes 130 is measured by the controller 140. That is, the controller 140 includes a calculator that calculates a length by comparing the capacitances of the main electrodes 120 and the reference electrodes 130

In this case, it is preferable that the pair of main electrodes 120 and the pair of reference electrodes 130 are made of the same material in the same shape, and the main electrodes 120 and the reference electrodes 130 have a curved surface or flat surface shape. However, it is advantageous that the main electrodes 120 and the reference electrodes 130 have a curved surface shape that can secure a space as much as possible in order to secure a space in which a laser travels.

In FIG. 3, when the reference electrodes 130 and the main electrodes 120 are inserted to a virtual surface 30 showing the skin surface of a body in the arrow direction 31 that means the inside of the skin (human tissue), the same permittivity is applied to the capacitance that is induced at the reference electrode section R inserted in the body and the capacitance that is induced at the main electrode section D1 inserted in the body, and the permittivity of the air is applied to the section D2 of the main electrodes 120 that is not inserted into the body. Since the permittivity of the air is a low value in comparison to the permittivity of a body, only the insertion depth of electrodes inserted into a body contributes to inducing capacitance, so it is possible to measure the insertion depth of the main electrodes 120. It is shown in FIG. 3 that the main electrodes 120 and the reference electrodes 130 are physically far from each other, but this is for helping understanding and the main electrodes 120 and the reference electrodes 130 may be disposed adjacent to each other even though they are physically separated.

In an embodiment of the present disclosure, when the pair of reference electrodes 130 is fully inserted in a body and the pair of main electrodes 120 is partially inserted into the body, and when the length of the reference electrodes 130 is B, the capacitance that is induced at the reference electrodes 130 is C1, the length of the section D1 of the main electrodes 120 inserted into the body is X, and the capacitance at the section of the main electrodes 120 inserted into the body is C2, the following equation is satisfied.

In FIG. 3, it is possible to calculate an insertion depth by measuring the capacitances induced at the reference electrodes 130 and the main electrodes 120 using a measurer 35.

$$X = ( {B}^{*} C 2 ) / C 1 \qquad ( 2 )$$

In this case, since the same permittivity is applied to the reference electrode section R inserted into the body and the main electrode section D1 inserted into the body, ε₀, εᵣ, and d are the same in the above equation (1). Assuming that the areas of the main electrodes 121 and 122 and the reference electrodes 131 and 132 have the same width, the areas of the main electrodes 121 and 122 and the reference electrodes 131 and 132 are proportioned to the length, so the equation (2) holds.

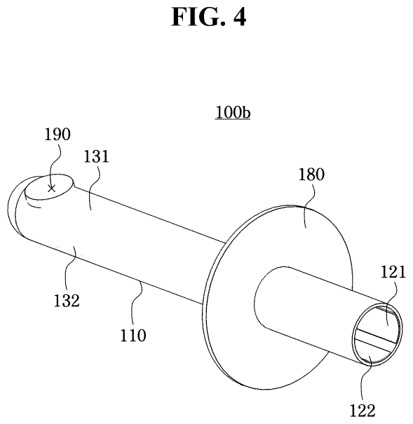

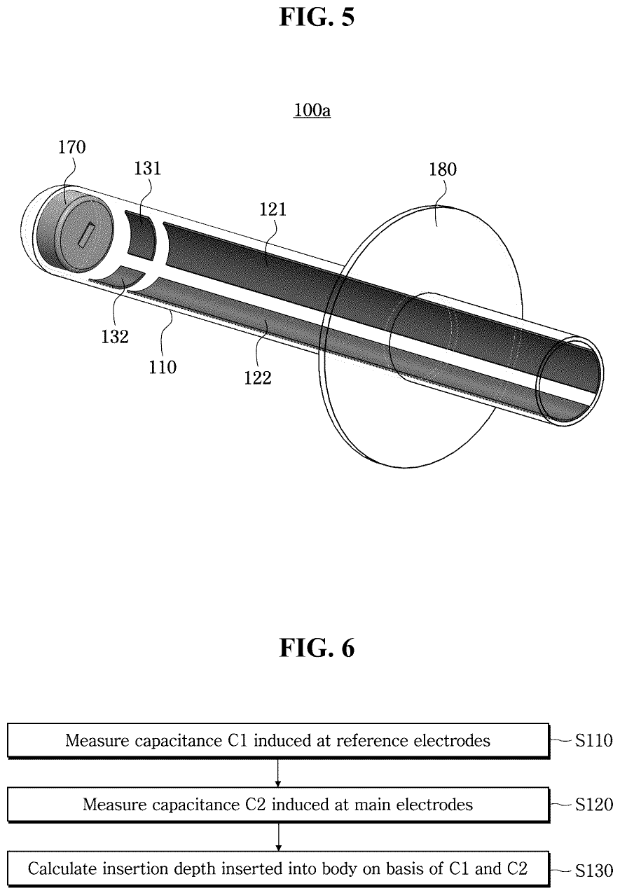

FIG. 4 is a view of a measuring apparatus 100a obtained by applying the non-metallic protective container 110 according to an embodiment of the present disclosure to a surgical instrument for a body, and FIG. 5 is a view of a measuring apparatus 100b when the non-metallic protective container 110 according to an embodiment of the present disclosure is applied to an endoscope.

In more detail, FIG. 4 is view showing the case when an insertion depth measuring apparatus 100a is applied to a female genital surgical instrument using a capacitance measurement technique, and FIG. 5 is a view showing the case when an insertion depth measuring apparatus 100b is applied to an endoscope. Referring to FIG. 4, the protective container 110 has a laser output window 190, so a laser can be output to the outside through the window 190. That is, a laser is emitted to a body through the laser output window 190 of the female genital surgical instrument after traveling through the protective container 110.

Further, referring to FIG. 4, it can be seen that a disc-shaped insertion separation plate 180 is disposed on the circumference of the protective container 110. The insertion separation plate 180 can prevent body fluid that may be produced in a surgery from flowing into the measuring apparatus 100.

In the related art, the insertion depth of surgical instruments was measured by marking a scale on the surface of vaginal surgical instruments and visually inspecting it by an operator. Referring to FIG. 4 and FIG. 5, in an embodiment of the present disclosure, it is possible to measure insertion depth of an insertion tube by installing the main electrodes 120, if necessary, installing the reference electrodes 130 in the insertion tube.

FIG. 5 shows the case when the capacitance measurement technique is applied to an endoscope. The protective container 110 may be an insertion tube into which a medical instrument for treating the inside of a body is inserted, and an endoscopic camera 170 may be disposed at the front end inside the protective container 110. That is, FIG. 5 shows the case when a camera 170 is installed in an insertion tube 51. In an embodiment of the present disclosure, in an insertion tube 51, the camera 170 is installed and the main electrodes 120 are installed, or if necessary, the reference electrodes 130 are installed, whereby it is possible to simultaneously obtain depth information at parts of an image.

Meanwhile, in an embodiment of the present disclosure, a method for measuring an insertion depth of medical instruments inserted into a body is provided. FIG. 6 is a flowchart of a method for measuring an insertion depth of medical instruments inserted into a body according to an embodiment of the present disclosure and the following description refers to FIG. 6.

The apparatus 100 for measuring an insertion depth of medical instruments inserted into a body is the same as the above description. That is, the measuring apparatus 100 includes: a non-metallic protective container 110 that is inserted into a body with a medical instrument disposed therein; a pair of main electrodes 120 having a predetermined area and formed in a longitudinal direction while facing each other in the protective container 110; and a controller 140 electrically connected to the pair of main electrodes 120, measuring capacitance that is induced by the pair of main electrodes 120, and calculating the insertion depth inserted into a body on the basis of the measured capacitance. That is, the apparatus 100 for measuring an insertion depth of medical instruments inserted into a body according to an embodiment of the present disclosure includes a protective container 110 protecting a medical instrument, a pair of capacitor electrodes that is main electrodes 120, and a controller 140 provided outside the protective container 110 and controlling the medical instrument. In FIG. 1 and FIG. 2, a medical instrument insertion hole is formed at the left portion of the protective container 110, so medical instruments can be inserted.

The medical instrument protective container 110 is made of non-metal and capacitance induced at the main electrodes 120 inserted into a body reflects permittivity of a body around the electrodes inserted in the body. The non-inserted portions of the main electrodes 120 are exposed to the air, so capacitance reflects the permittivity of the air.

In order to measure the insertion depth of the medical instrument inserted into a body, the measurement is performed by including the steps of first, measuring capacitance C1 in the body by a pair of reference electrodes 130 disposed in the non-metallic protective container 110 that is inserted into the body with a medical instrument disposed therein (S110), measuring capacitance C2 induced at a pair of main electrodes 120 disposed on extension lines of the reference electrodes 130 in the protective container 110 (S120), and calculating the insertion depth of the main electrodes 120 inserted into the body using C1 and C2 (S130). In this case, the permittivity C2 changes, depending on the insertion depth in the body.

If the length of the reference electrodes 130 is B and the length of the section of the main electrodes 120 that is inserted into a body is X, the length of the section of the main electrodes 120 that is inserted into the body can be obtained from the above equation (2).

The detailed description should not be construed as being limited in all respects and should be construed as an example. The scope of the present disclosure should be determined by reasonable analysis of the accompanying claims, and all changes within an equivalent range of the present disclosure are included in the scope of the present disclosure.

What is claimed is:

1. An apparatus for measuring an insertion depth of medical instruments inserted into a body, the apparatus comprising:
a non-metallic protective container that is inserted into a body with a medical instrument disposed therein;
a pair of main electrodes having a predetermined area and formed in a longitudinal direction while facing each other in the protective container; and
a controller electrically connected to the pair of main electrodes, measuring capacitance that is induced by the pair of main electrodes, and calculating an insertion depth of the medical instrument inserted into a body on the basis of the measured capacitance.

2. The apparatus of claim 1, further comprising:
a pair of reference electrodes disposed on extension lines of the main electrodes, formed electrically and separately from the main electrodes, facing each other, and having a preset length,
wherein capacitance that is induced at the reference electrodes is measured by the controller.

3. The apparatus of claim 2, wherein the pair of main electrodes and the pair of reference electrodes are made of the same material in the shape.

4. The apparatus of claim 3, wherein when the pair of reference electrodes is fully inserted in a body and the pair of main electrodes is partially inserted into the body, and when a length of the reference electrodes is B, capacitance that is induced at the reference electrodes is C1, a length of the section of the main electrodes inserted into the body is X, and capacitance at the section of the main electrodes inserted into the body is C2, the following equation is satisfied:
X=(B*C2)/C1.

5. The apparatus of claim 2, wherein the main electrodes and the reference electrodes have a curved surface or flat surface shape.

6. The apparatus of claim 1, wherein the protective container is an instrument that treats the inside of a body, and
an endoscopic camera is disposed at a front end inside the protective container.

7. The apparatus of claim 1, wherein a disc-shaped insertion separation plate is disposed on a circumference of the protective container.

8. The apparatus of claim 1, wherein the protective container has a laser output window, so a laser is output to the outside through the window.

9. A method for measuring an insertion depth of medical instruments inserted into a body, the method comprising:
measuring capacitance C1 in a body by a pair of reference electrodes disposed in a non-metallic protective container that is inserted into the body with a medical instrument disposed therein,
measuring capacitance C2 induced at a pair of main electrodes disposed on extension lines of the reference electrodes in the protective container; and
calculating an insertion depth of the main electrodes inserted into the body using the C1 and C2,
wherein the permittivity C2 changes, depending on the insertion depth inserted into the body.

10. The method of claim 9, wherein when length of the reference electrodes is B, and a length of the section of the main electrodes that is inserted into a body is X, an insertion depth of the main electrodes inserted into the body is obtained from the following equation:
$$X = ( {B}^{*} C 2 ) / C 1.$$
